(19) United States
(12) Patent Application Publication
(10) Pub. No.: US 2023/0144577 A1
(43) Pub. Date: May 11, 2023

(54) AIR DISINFECTION DEVICE AND METHOD USING SAME

(71) Applicant: Q One Holding AG, Sarnen (CH)

(72) Inventor: Bernd Hoehne, Achim (DE)

(21) Appl. No.: 17/913,496

(22) PCT Filed: Apr. 1, 2021
(86) PCT No.: PCT/EP2021/058661

§ 371 Date: Sep. 22, 2022

(30) Foreign Application Priority Data
Apr. 6, 2020 (DE) 10 2020 109 578.3

(51) Int. Cl. A61L 9/20

(52) U.S. Cl. CPC A61L 9/20; A61L 2209/12; A61L 2209/14; A61L 2209/111

(57) ABSTRACT

An air disinfection device wherein air to be disinfected flows through a disinfection chamber (1) from an air inlet (11) to an air outlet (12), wherein one or more UVC lasers (21) are provided as UVC light source means (2), and a multiplicity of mirrors (17) are provided as a reflective surface in the interior (10) of the disinfection chamber (1), arranged in such a way that one or more UVC laser beams (26) emitted by the UVC laser (21) pass through the interior (10) of the disinfection chamber (1) so as to be reflected multiple times. Also, a method for disinfecting air using this device.

The invention relates to an air disinfection device with a disinfection chamber with an air inlet, an interior and an air outlet, wherein the air to be disinfected flows through the disinfection chamber from the air inlet to the air outlet, wherein UVC light sources are provided in the disinfection chamber and the interior of the disinfection chamber (1) is equipped with a reflective surface. Furthermore, the invention relates to an air disinfection method with a disinfection chamber with an air inlet, an interior and an air outlet, wherein the air to be disinfected flows through the disinfection chamber from the air inlet to the air outlet and is exposed to UVC irradiation, in particular with an air disinfection device.

Air disinfection devices are known in the prior art in various embodiments and for various applications. For example, so-called air purifiers are known for living rooms that have an air inlet and an air outlet with an intermediate sterilization chamber. The sterilization chamber contains air purifiers, often a filter or air scrubber or UV light irradiation. However, such air purifiers are usually require house electrical power and accordingly are not mobile or are very unwieldy to use. Furthermore, it is difficult to use the known air purifiers for particularly strict hygienic requirements, for example in hospitals, retirement homes and especially for individual persons, since the respective air flow is only incompletely exposed to the germicidal UVC light irradiation.

Furthermore, there are respirators, which also have a kind of cleaning chamber, in which germ-retaining and/or germicidal material (filter material) is contained, through which the wearer of the mask breathes in ambient air when inhaling. In the case of such masks, exhalation valves are often provided in order to enable a controlled path for the air exhaled by the wearer and to avoid that the mask lifts off when the wearer exhales, and so possibly drawing in external air. On the other hand, technically complex air preparation and disinfection devices are known, which are known, for example, for inpatient use in hospitals.

DE 10 2018 129 811 A1 describes a pressure vessel for receiving compressed air for dental applications, wherein the compressed air is disinfected by means of UVC LEDs with internal reflection surfaces arranged in the container. The compressed air vessel can be regarded as a disinfection chamber, whereby only one passage is provided for the supply and discharge of air.

DE 200 21 236 U1 describes a device for purifying air in which an air flow from a lower air inlet to an upper air outlet is routed through a housing with UVC light sources, wherein the light sources within the housing, which consists of two parabolically curved areas, are arranged in the respective focal points (focal lines).

In WO 03/039604 A2, an air disinfection device is shown, which has an air inlet and an air outlet with a disinfection chamber arranged in between. The UV light source is arranged outside the disinfection chamber in the embodiment shown and radiates through a UV light inlet opening into the disinfection chamber. This chamber is equipped with partially reflective surfaces and a scattering mirror. An air inlet tube is made of UV-permeable quartz glass to distribute the UV light within the housing and, when coated appropriately, fully reflect it.

Based on this prior art, it is an object of the invention to provide an air disinfection device or a method with which a reliable disinfection of the air flowing through it, in particular for the breathing air supply for persons to be protected from infections, is made possible, wherein the device is permanently usable, cost-effective, small-scale, energy-efficient and thus in particular mobile.

This object is achieved with an air disinfection device according to claim 1 and a method according to claim 11. The preferred use of this device or this method for air disinfection is the supply of persons to be protected from infections and/or the disinfection of exhaled air of an infectious person.

As a result of having as UVC light source means (2) one or more UVC lasers (21) as well as a large number of mirrors (27) provided as a reflective surface in the interior (10) of the disinfection chamber (1), wherein the UVC laser(s) (21) and mirrors (17) are arranged in such a way that UVC laser beam(s) (26) emitted by the UVC laser(s) (21) pass multiple times as reflections through the interior (10) of the disinfection chamber (1), and that the disinfection chamber (1) is cuboidal, wherein the disinfection chamber (1) has a height (H) that corresponds to the diameter of a single UVC laser beam (26) or to the vertical extent in the case of several UVC laser beams (26) located one above the other, the intense UVC radiation from the UVC laser or lasers is distributed throughout the interior of the disinfection chamber, so that the air flowing through the disinfection chamber and thus any material in the air, in particular any microorganisms, are exposed to the disinfecting UVC irradiation over a longer exposure path and a longer exposure time, namely the length of time in which an air particle flows through the disinfection chamber from the air inlet to the air outlet. Accordingly, a good killing effect against microorganisms, bacteria and/or viruses is achieved and thus the bacterial count in the transmitted air is significantly reduced. Furthermore, a flat- or planar-shaped disinfection chamber is provided, which is exposed in each plane by a respective UVC laser beam. In the case of a greater height of the disinfection chamber, several UVC lasers can also be arranged stacked on top of each other in such a way that their laser beams propagate in different, parallel levels and fill the entire flat-cuboid disinfection space. For this purpose even UVC lasers with a low power of a few milliwatts are sufficient to disinfect an average breathing volume of a person when flowing through the disinfection chamber. In particular, the UVC laser radiation selectively allows higher energy to act on the radiated medium.

If the UVC laser(s) are arranged on the outside of the disinfection chamber, with the laser output at which the laser beam is decoupled pointing through an opening into the disinfection chamber, the laser does not affect the air passage in the interior of the disinfection chamber. Furthermore, the laser is easier to replace for maintenance purposes.

As a result of the fact that, in the case of a single UVC laser beam, the UVC laser beam is coupled into the disinfection chamber in a central plane, which is oriented centrally and perpendicular to the height extension of the cuboid disinfection chamber, and the mirrors are oriented perpendicular to this central plane, an alignment of the introduced laser beam centrally within the disinfection chamber along the central plane is ensured despite multiple reflections and therewith an effective disinfection in the interior of the disinfection chamber, which has been painted over by the laser beam, has been achieved.

In the case of several UVC laser beams arranged one above the other, the UVC laser beams are coupled parallel to the central plane in the disinfection chamber, whereby the UVC laser beams are distributed equidistantly along the height extension. Accordingly, the entire interior of the disinfection chamber is covered even with somewhat greater height extension. In this respect, a high efficiency of the UVC irradiation in the disinfection chamber, and thus an optimal disinfection of the air flowing through it, is achieved.

If the mirrors are secured in a fixed arrangement in the interior, a very robust disinfection chamber can be provided. For example, the disinfection chamber is formed as a one-piece cast aluminum component with internally polished mirror surfaces, which forms a particularly stable disinfection chamber, which shows a high consistency and permanence with regard to the orientation of the mirrors for the laser beam(s).

A further increase in efficiency is achieved for a given UVC radiation power by the fact that the walls of the disinfection chamber facing the interior are mirrored. Scattered light from the UVC laser beams is thus repeatedly reflected back into the interior of the disinfection chamber and improves the UVC light penetration and thus the disinfecting effect.

Preferably, the UVC laser is an LED laser that emits UVC radiation with wavelengths of 200 nm to 280 nm, in particular 250 nm to 270 nm. UVC-LED lasers with a power of 1 mW to 70 mW, in particular 3 mW to 20 mW, can be used, which are available on the market at reasonable prices.

In order to keep the interior of the disinfection chamber as dust-free as possible and to keep the breathing air to be disinfected as free as possible from suspended dirt particles, a particulate filter and/or a controllable inlet valve are provided at the air inlet. With the controllable inlet valve, the air inlet can be opened only as required, which can reduce the possible entry of dirt.

If a check valve and/or a controllable exhaust valve are provided at the air outlet, the check valve at the air outlet prevents air from flowing back through the disinfection chamber in the direction opposite of intended. Alternatively or additionally, the controllable exhaust valve can take over this function or also block the air flow during breaks in use.

In a further development, a first biosensor is provided at the air inlet and/or a second biosensor at the air outlet. With a biosensor at the air inlet, the biological quality of the supplied air, in particular the presence of microorganisms, can be determined. A second biosensor can also measure the biological quality of the air in addition or alternatively at the air outlet. Thus, when using the air disinfection device for a person to be protected, it is possible to measure the content of any microorganisms in the air before the disinfection device and when leaving the disinfection device in order to be able to prove the quality of the disinfection. Conversely, even when using the air disinfection device with an infectious person, the exhaled air can be checked, so that the still possible presence of microorganisms can first be checked at the air inlet via the first biosensor and the proper germ reduction checked by the second biosensor at the air outlet.

Accordingly, a process-appropriate solution is characterized by the steps: generating at least one UVC laser beam, which is directed into the interior of the disinfection chamber in a central plane that is aligned perpendicular to the height extension of the cuboid disinfection chamber, wherein the height of the disinfection chamber corresponds to the diameter of the UVC laser beam or the UVC laser beams arranged one above the other, reflecting the UVC laser beams multiple times in the interior, whereby the UVC laser beams essentially pass through the entire interior of the disinfection chamber parallel to the central plane.

Due to the distribution of the UVC laser beams with multiple reflections in the interior of the disinfection chamber, essentially the entire interior of the disinfection chamber is covered, so that flowing air is intensively penetrated by the UVC laser beams, so that any microorganisms present in it are reliably killed, i.e. the bacterial count is significantly reduced.

In the case that each UVC laser beam is absorbed after passing through the interior of the disinfection chamber, precisely definable paths are provided for each UVC laser beam and uncontrollably scattered UVC laser light is avoided by the absorption at the end of the run.

In an alternative version, each UVC laser beam is coupled back to itself at the beginning of its path after passing through the interior of the disinfection chamber. This ensures that the residual intensity of the respective UVC laser beam after passing through the interior is not destroyed by absorption, but coupled to amplify the signal.

In a further alternative embodiment several laser beams are generated, wherein onto a first laser beam, after a partial running path, several times in succession an additional laser beam is introduced coupled to the laser beam. This means that there are several UVC lasers in a row, which emit their UVC radiation on a common light path, but between two successive lasers there is a partial path for the UVC laser beam, on which a certain attenuation of the signal takes place, whereby further UVC radiation energy is added by each additional laser. At the end of the path of this amplified cascading laser beam, an absorption at a defined point as well as a feedback of the laser beam to the beginning of its laser path can take place.

An advantageous signal amplification of the UVC radiation is achieved in particular if the UVC laser beam is coupled in the same phase after the run. This can be achieved by precisely defining the path length for the UVC laser beam at its wavelength and coherence length up to the recouplement point.

If the UVC laser beams are guided crossing-free in the interior of the disinfection chamber, a largely interference-free course of the UVC laser beams is achieved over the entire route. This essentially ensures the effectiveness of UVC radiation for killing microorganisms over the entire path of UVC radiation. The attenuation of the signal by scattering/dissipation on air particles is likely to remain very low.

Alternatively, the UVC laser beams intersect on their path in the interior of the disinfection chamber. In this design, a mutual influence of intersecting laser beams is deliberately accepted in order to enable a greater distribution of UVC radiation in the interior of the disinfection chamber. A lower construction accuracy for the disinfection chamber is permissible, so that it can be created more cost-effectively. Of course, the attenuation of the signal intensity by scattering and superposition phenomena is greater than with the crossing-free guidance of the UVC laser beams according to the embodiment described above. However, the entire air volume passed through the disinfection chamber is likely to be completely illuminated by this significantly more scattered UVC radiation, so that any occurring, isolated germs (microorganisms) are in any case touched by UVC radiation, which is not always to be expected in the version shown above due to the very discreet propagation of the UVC laser beams.

When pulsed UVC laser beams are used, high-energy UVC laser pulses can be introduced into the interior of the disinfection chamber, which in turn have a strong germicidal effect. Due to the multiple reflections of these pulsed UVC laser beams, in turn, essentially the entire interior of the disinfection chamber is passed through, whereby in certain geometric constellations intersecting paths of the specified UVC laser beams are also possible, without the energetically rich UVC laser light pulses meeting at these crossing points.

In the following, five embodiments of the invention are described in detail on the basis of the enclosed drawings.

There is shown in:

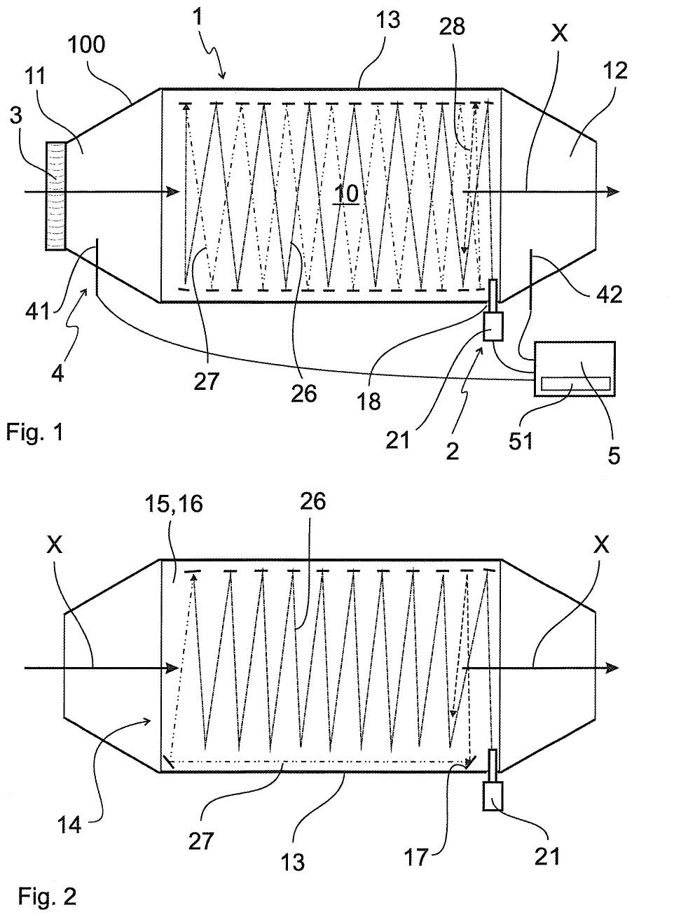

FIG. 1 an air disinfection device according to a first embodiment in a schematic, partly cut top view,

FIG. 2 an air disinfection device in a second embodiment,

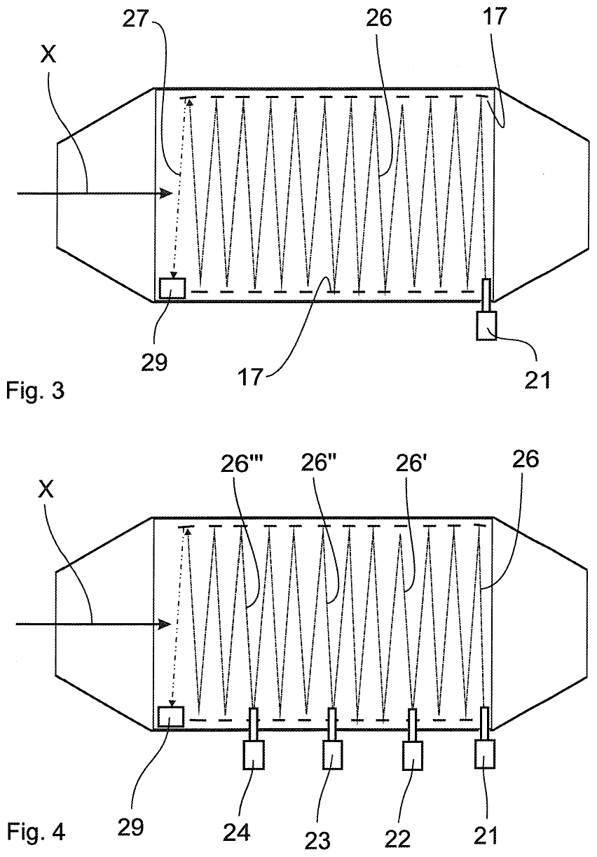

FIG. 3 an air disinfection device in a third embodiment and

FIG. 4 an air disinfection device in a fourth embodiment.

In FIG. 1, a first embodiment of an air disinfection device is shown in a schematic, partially sectional view. The air disinfection device has a disinfection chamber 1, wherein the disinfection chamber 1 has an interior 10, to which on one side of the disinfection chamber 1 an air inlet 11 and on the opposite side an air outlet 12 are arranged. Air can thus flow through this disinfection chamber 1 from air inlet 11 through interior 10 to air outlet 12.

In the embodiment shown here, the disinfection chamber 1 is essentially rectangular, wherein the largest longitudinal extension is aligned in the flow direction X of the air flowing through the disinfection chamber 1 and the width of the disinfection chamber 1 is aligned transversely to the flow direction X of the air. In FIG. 1, the side walls 13 of the disinfection chamber 1 are shown above and below in the drawing plane. Front sides 14 of the disinfection chamber 1 are shown in drawing level in FIG. 1 on the left and right. The front sides 14 are open for a free flow of the air to be disinfected. Around the disinfection chamber 1 a housing 100 may be arranged, which for the air inlet 11 and air outlet 12 is provided suitable transitions to hose connections etc. for the conduction of the air flowing to the air disinfection device for disinfection, or for the conduction of the disinfected air emitted by the air disinfection device.

In FIG. 1, the disinfection chamber 1 is shown in top view, i.e. omitting the top 15 to permit a view of the bottom 16. In the disinfection chamber 1, 13 mirrors 17 are arranged in the interior 10 along the two side walls. Furthermore, an opening 18 is provided at the corner of the disinfection chamber 1 shown at the bottom right in FIG. 1, at which a first UVC laser 21 is attached as UVC light source 2. The laser output, at which a UVC laser beam 26 is decoupled, is directed into the opening 18, so that the UVC laser beam 26 is directed into the interior 10 of the disinfection chamber 1. Preferably, the first UVC laser 21 with its laser output in the opening 18 is formed airtight.

The UVC laser beam 26, which is emitted by the first UVC laser 21, is shown as a dot-dash line. The UVC laser beam 26 is reflected several times over its path on the mirrors 17, so that a path of the UVC laser beam 26 (dashed representation) extends over the entire interior 10 of the disinfection chamber 1. At the end of the disinfection chamber 1 shown on the left in FIG. 1 in the drawing plane, the UVC laser beam 26 is reflected back into a returning laser beam 27 (marked with double dot dash), so that a corresponding return of the laser beam results, which is appropriately coupled back to itself at the beginning of its path (see short-dashed coupling laser beam 28).

In the embodiment shown here, the travel of the UVC laser beam 26, returning laser beam 27 and coupling laser beam 28 is on a plane parallel to the top 15 or bottom 16 of the disinfection chamber 1, so that the paths of the UVC laser beam 26 intersect with the paths of the returning laser beams 27 and coupling laser beam 28. At these crossing points, a deliberate scattering or turbulence of the UVC radiation takes place, so that an increased distribution of UVC radiation in the interior 10 of the disinfection chamber 1 is to be expected.

FIG. 2 shows a second embodiment of the invention in which the same or similar components to the embodiment according to FIG. 1 are designated with the same reference symbols. However, there is a significant difference in the path of the laser beams 26, 27, 28, since here a crossing-free design of the path of the laser beams 26, 27, 28 results from the fact that the returning laser beam 27 is directed into a plane below or above the plane in which the UVC laser beam 26 is oriented within the interior 10 via correspondingly oriented mirrors. This ensures that the returning laser beam 27 is coupled back to the beginning of its path (UVC laser beam 26) via appropriately aligned mirrors 17 by means of a coupling laser beam 28.

In FIG. 3, in a third embodiment, an air disinfection device with a disinfection chamber 1 is shown, in which the UVC laser beam 26 emitted by the first UVC laser 26 is guided along a plane parallel to the drawing plane through the interior 10 of the disinfection chamber 1 to a laser beam absorber 29 at the left end of the disinfection chamber 1 in the drawing plane. Also in this design, the UVC laser beam 26 is crossing-free.

As in the other embodiments, the clear height of the interior 10 of the disinfection chamber 1 should be designed in such a way that it corresponds as exactly as possible to the radiation diameter of the first UVC laser 21. This ensures that the entire free cross-section according to the open front sides 14 over the entire area of the interior 10 is passed through by the UVC laser beam 26 and, if necessary, by the returning laser beam 27 (according to embodiments according to FIGS. 1 and 2). This ensures that the air flowing through the interior 10 of the disinfection chamber 1 interacts with the UVC radiation and, in particular, microorganisms carried in the air are killed by the UVC radiation.

In a fourth embodiment according to FIG. 4, a total of four UVC lasers 21, 22, 23, 24 are provided as UVC light source 2, respectively arranged at four corresponding, spaced openings 18 on the disinfection chamber 1. The first UVC laser 21 generates a UVC laser beam 26, which is absorbed into a newly introduced UVC laser beam 26′ after a partial run path, so that the intensity of the attenuated UVC laser beam of the first UVC laser 21 is amplified with the newly introduced UVC laser beam 26′. After a further partial route, a new UVC Laser beam 26″ is then coupled to this UVC laser beam 26′ from a third UVC laser 23. This in turn amplified UVC laser beam signal is now mirrored several times over a further partial path until the UVC laser beam merges again with a new UVC laser beam 26″′, generated from a fourth laser 24. This UVC laser beam 26″′ is then directed to a laser beam absorber 29 after a subsequent partial path.

Of course, this embodiment could also include a returning laser beam 27 with renewed coupling at the beginning of the path. Of course, further combinations of the aforementioned features from the four embodiments can be combined with each other in order to be able to specify further embodiments of the invention.

Furthermore, further features of the air disinfection device are now explained vicariously on the basis of the embodiment according to FIG. 1. These features can also further develop the embodiments of FIGS. 2 to 4 and modified embodiments.

On the air disinfection device shown in FIG. 1, a particulate filter 3 is provided at the air inlet 11, which frees the air flowing in at the air inlet 11 from suspended particles and dirt particles, so that the interior 10 of the disinfection chamber 1 remains as clean as possible, and with the UVC irradiation of the air flowing through the interior 10 the lowest possible radiation losses occur due to scattering and dissipation. Furthermore, an inlet valve may be provided at inlet 11. On the one hand, this inlet valve can be a simple mechanical check valve to prevent air flow against the desired inlet direction (flow direction X). Alternatively, the inlet valve can also be an electrically controllable valve. An exhaust valve is also provided at air outlet 12. That can also be designed as a check valve to avoid an undesirable return flow of air against the flow direction X or as a controllable exhaust valve.

In further embodiment, sensors 4 are provided, which can measure conditions at the air disinfection device. Preferably, a first biosensor 41 may be provided at the air inlet 11 and a second biosensor 42 at the air outlet 12. Furthermore, sensors 4 can be air flow meters, for measuring the ozone content of the air after UVC irradiation and pressure sensors.

The sensors 4 are connected to a control unit 5, which preferably has a rechargeable battery 51 as a power supply. The UVC lamps 2 are connected to the control unit 5. The UVC light sources can, for example, light up continuously during the use of the air purification device (plugged in battery). Alternatively, the UVC-LED laser can also be switched on only for the necessary disinfection process, for example for each breath (triggering via pressure sensors). Furthermore, in the case of corresponding controllable valves, namely the inlet valve and/or the exhaust valve may be connected to the control unit 5.

In the following, the air disinfection method is described on the basis of the embodiments proposed here according to FIGS. 1 to 4 of the air disinfection device.

For example, air to be disinfected is sucked in by a person to be protected from infection via a breathing mask arranged at the air outlet 12. During this process, the UVC light source agents 2 are switched on via control unit 5, so that in the interior 10 of the disinfection chamber 1 UVC laser beams 26, 27, 28 essentially pass through the entire interior 10 of the disinfection chamber 1. Now the ambient air sucked in by the person is directed via the air inlet 11 into the interior 10. If necessary, a particulate filter 3 at the air inlet 11 can free the air thus sucked in from suspended dust and dirt particles. There, the UVC radiation of the UVC laser beams 26, 27, 28 interacts with the particles in the air, in particular germs and microorganisms are killed. The air disinfected in this way now reaches the breathing mask of the person to be protected via the air outlet 12, which is not shown here.

Alternatively, with the appropriate formation of the air disinfection device, both inhalation and exhalation can be passed directly through the one disinfection chamber 1. It must be taken into account that the air volume in the interior 10 of the disinfection chamber 1 is small compared to the normal breathing volume of a person, so that always sufficient fresh ambient air leads to the person to be ventilated. In this case, both the inhaled air and the exhaled air can be disinfected advantageously with only one air disinfection device, ie with only one disinfection chamber 1, so that the exhaled air of an infectious person can also be disinfected.

In further process-appropriate formation, two air disinfection devices, ie two disinfection chambers 1 may also be arranged in parallel, one by means of suitable valves, in particular check valves only for inhalation and the other disinfection chamber 1 is only responsible for exhalation. In this version, larger volumes can also be used in the disinfection chamber 1, since the problems of an air column only moving back and forth without sufficient exchange with fresh air are not to be feared.

To control the efficiency and quality of disinfection, corresponding biosensors 41, 42 can also measure the quality of the supplied or discharged air. In addition, pressure sensors may also be provided, for example, to immediately detect a small negative pressure when inhaling through a breathing mask connected to the air outlet 12, in order to then be able to open appropriately controllable inlet and outlet valves. Furthermore, the amount of air can also be measured, for example, to increase the light intensity of the UVC light source agents 2 in a faster and/or stronger breathing or to reduce the radiation intensity in a calmer breathing. As a further quality check, an ozone sensor may be provided, which gives an alarm signal if an ozone limit value is exceeded or reduces the intensity of the UVC radiation. However, this should not be necessary for UVC radiation in the wavelength range of 250 to 270 nm, since ozone is more likely to form at wavelengths below 200 nm when UVC is irradiated by air.

Advantageously, the air disinfection device with its UVC-LED laser can reliably disinfect air for inhalation or exhalation, whereby due to the low energy requirement of the UVC-LED laser a mobile use with handy rechargeable batteries (for example, power pack for mobile phones) for a long period of several hours or days is possible. After changing the rechargeable battery or recharging the battery the air disinfection device can always be reused. This is a considerable advantage over disposable protective masks, which can only be used for a maximum period of use of a few hours and must subsequently be disposed of.

Reference list

| | |
|---|---|
| 1 | Disinfection chamber |
| 10 | Interior |
| 100 | Casing |
| 11 | Air inlet |
| 12 | Air outlet |
| 13 | Sidewall |
| 14 | Face |
| 15 | Top |
| 16 | Subside |
| 17 | Mirrors |
| 18 | Opening |
| 2 | UVC Light source means |
| 21 | First UVC laser |
| 22 | Second UVC laser |
| 23 | Third UVC laser |
| 24 | Fourth UVC laser |
| 26, 26′, 26″, 26″′ | UVC laser beams |
| 27 | Returning laser beam |
| 28 | Launch laser beam |
| 29 | Laser beam absorber |
| 3 | Particle filter |
| 4 | Sensor |
| 41 | First biosensor |
| 42 | Second biosensor |
| 5 | Control unit |
| 51 | Rechargeable battery |
| X | Flow direction |

1. An air disinfection device with a disinfection chamber (1) with an air inlet (11), an interior (10) and an air outlet (12), wherein air to be disinfected flows through the disinfection chamber (1) from the air inlet (11) to the air outlet (12), wherein UVC light source means (2) are provided in the disinfection chamber (1) and the disinfection chamber (1) is equipped in the interior with reflective surface(s), wherein as UVC light source means (2) a UVC laser (21) is provided and as reflective surface in the in the interior (10) of the disinfection chamber (1) a number of mirrors (17) are provided, wherein the UVC laser (21) and mirrors (17) are arranged in such a way that UVC laser beam(s) (26) emitted by the UVC laser (21) are reflect multiple times in the interior (10) of the disinfection chamber (1), wherein the disinfection chamber (1) is cuboid shaped, the UVC laser beam (26) is coupled in the disinfection chamber in a central plane (F), which is centered and perpendicular to the height extension of the cuboid disinfection chamber (1), the mirrors (17) are oriented perpendicular to this central plane (F), and wherein the disinfection chamber (1) has a height (H) corresponding to the diameter of a single UVC laser beam (26).

2. An air disinfection device with a disinfection chamber (1) with an air inlet (11), an interior (10) and an air outlet (12), wherein air to be disinfected flows through the disinfection chamber (1) from the air inlet (11) to the air outlet (12), wherein UVC light source means (2) are provided in the disinfection chamber (1) and the disinfection chamber (1) is equipped in the interior with reflective surface(s), wherein as UVC light source means (2) several UVC lasers (21) are provided and as a reflective surface in the interior (10) of the disinfection chamber (1) a number of mirrors (17) are provided, wherein the UVC lasers (21) and mirrors (17) are arranged in such a way that UVC laser beams (26) emitted by the UVC lasers (21) are reflect multiple times in the interior (10) of the disinfection chamber (1), wherein the disinfection chamber (1) is cuboidal, wherein the UVC laser beams (26) are coupled in the disinfection chamber parallel to a central plane (F), which is centered and perpendicular to the height extension of the cuboid disinfection chamber (1), and wherein the disinfection chamber (1) has a height (H) corresponding to the height of the stacked laser beams (26).

3. An air disinfection device according to claim 1, wherein the UVC laser(s) (21) are arranged on the outside of the disinfection chamber (1), wherein the laser output, at which the laser beam (26) is decoupled, is directed into the disinfection chamber (1) through opening (18).

4. An air disinfection device according to claim 1, wherein the mirrors (17) are fixed in a fixed arrangement in the interior (10).

5. An air disinfection device according to claim 1, wherein the walls of the disinfection chamber (1) facing the interior (10) are mirrored.

6. An air disinfection device according to claim 1, wherein the UVC laser (21) is an LED laser that emits UVC radiation with wavelengths in the range of 200 nm to 280 nm.

7. An air disinfection device according to claim 1, wherein at the air inlet (11) a particulate filter (3) and/or a controllable inlet valve are provided.

8. An air disinfection device according to claim 1, wherein at least one of a particulate filter (3) and a controllable inlet valve are provided at the air inlet (11).

9. An air disinfection device according to claim 1, wherein at the air inlet (11) an inlet biosensor (41) and/or at the air outlet (12) an outlet biosensor (42) are provided.

10. An air disinfection method for disinfecting air in a disinfection chamber (1) with an air inlet (11), an interior (10) and an air outlet (12), wherein the air to be disinfected flows through the disinfection chamber (1) from the air inlet (11) to the air outlet (12) and is exposed to UVC irradiation from one or more UVC laser beam(s) (26), wherein the disinfection chamber (1) is a cuboid disinfection chamber (1) with a height (H) measured in a height extension claim 1, the method comprising:
generating one or more UVC laser beams(s) (26) directed into the interior (10) of the disinfection chamber (1) along a central plane (F) aligned perpendicular to the height extension of the cuboid disinfection chamber (1), wherein the height (H) of the disinfection chamber (1) corresponds to the sum of the diameter(s) of the one or more UVC laser beam(s) (26),
reflecting the UVC laser beams (26) multiple times in the interior (10), whereby the UVC laser beams (26) essentially pass through the entire interior (10) of the disinfection chamber (1) parallel to the central plane.

11. An air disinfection method according to claim 10, wherein each UVC laser beam (26) is absorbed after passing through the interior (10) of the disinfection chamber (1).

12. An air disinfection according to claim 10, wherein each UVC laser beam (26) after passing through the interior (10) of the disinfection chamber (1) is coupled back to itself at the beginning of its path.

13. An air disinfection method according to claim 10, wherein multiple laser beams (26′, 26″, 26″′) are generated, wherein added onto a first laser beam (26) after a partial running path several times in succession at least one other laser beam (26′, 26″, 26″′) is coupled to the laser beam (26, 26′, 26″, 26″′).

14. An air disinfection method according to claim 12, wherein the UVC laser beam (26) is coupled in-phase after the run.

15. An air disinfection method according to claim 10, wherein the UVC laser beams (26) no not cross in the interior (10) of the disinfection chamber (1).

15. An air disinfection method according to claim 10, wherein the UVC laser beams (26) intersect along their path in the interior (10) of the disinfection chamber (1).

17. An air disinfection method according to claim 10, wherein pulsed UVC laser beams (26) are used.

18. (canceled)

19. An air disinfection device according to claim 1, wherein the UVC laser (21) is an LED laser that emits UVC radiation with wavelengths in the range of 250 nm to 270 nm.
